(19) United States
(12) Patent Application Publication
(10) Pub. No.: US 2017/0086401 A1
(43) Pub. Date: Mar. 30, 2017

(54) METHOD FOR PRODUCING THREE-LINE HYBRID WHEAT PARENT FOUNDATION SEEDS

(71) Applicant: Xinjiang Academy of Agri-Reclamation Sciences, Shihezi City (CN)

(72) Inventors: Xiaoming TIAN, Shihezi City (CN); Hongjun XU, Shihezi City (CN); Yingbin NIE, Shihezi City (CN); Peiyuan MU, Shihezi City (CN); Xinnian HAN, Shihezi City (CN); Wei SANG, Shihezi City (CN); Fengjuan CUI, Shihezi City (CN)

(73) Assignee: Xinjiang Academy of Agri-Reclamation Sciences, Shihezi City (CN)

(21) Appl. No.: 15/372,383

(22) Filed: Dec. 7, 2016

Related U.S. Application Data
(63) Continuation of application No. PCT/CN2016/076973, filed on Mar. 22, 2016.

(30) Foreign Application Priority Data
Apr. 1, 2015 (CN) 201510153520.0

(51) Int. Cl. A01H 1/02

(52) U.S. Cl. CPC A01H 1/02

(57) ABSTRACT

Disclosed is a method for producing three-line hybrid wheat parent foundation seeds, including: determining whether sterile-line head progeny rows satisfy 100% sterile degree index of a sterile line or not by bagged selfing of the sterile-line head progeny rows, separately harvesting the bagged outcrossed heads satisfying the index to achieve cyclic breeding of sterile-line spike lines, and mixedly harvesting the sterile-line head progeny rows satisfying the index for sterile-line foundation seed plots; conducting test crossing on the separately harvested bagged outcrossed head seeds and the restorer-line head progeny rows, correspondingly numbering the restorer-line head progeny rows and bagged test-crossed heads, and separately harvesting bagged test-crossed seeds; conducting yield comparison on the separately harvested bagged test-crossed seeds, taking a part of preserved restorer-line head progeny row seeds meeting the requirement for cyclic breeding of restorer-line spike lines, and then planting the rest in restorer-line foundation seed plots.

### CROSS REFERENCE TO RELATED APPLICATIONS

This application is a continuation of International Application No. PCT/CN2016/076973, filed on Mar. 22, 2016, which is based upon and claims priority to Chinese Patent Application No. 201510153520.0, filed on Apr. 1, 2015, the entire contents of which are incorporated herein by reference.

### TECHNICAL FIELD

The present disclosure relates to the technical field of wheat breeding, and more particularly, to a method for producing three-line hybrid wheat parent foundation seeds.

### BACKGROUND

Wheat is a food crop maximum in total yield and high in nutritive value in the world. With further increase of population, the area of per capita cultivated land is decreased year by year, so that food safety is the first problem faced by the mankind. Hybrid vigor is a universal phenomenon in the living nature, a main measure for improving wheat yield is utilization of the hybrid vigor of wheat, and a ‘three-line method’ is one of main ways of utilizing the hybrid vigor of the wheat.

The three-line method refers to matching of a sterile line, a maintainer line and a restorer line, the sterile line serves as a female parent and the maintainer line serves as a male parent for interplanting, the sterile line is pollinated by the maintainer line, the next generation of seeds produced by the sterile line is still male sterile and used for continuous breeding of the sterile line and producing hybrid seeds with the restorer line; the sterile line serves as the female parent and the restorer line serves as the male parent for interplanting, the sterile line is pollinated by the restorer line, and seeds produced on sterile line plants are the hybrid seeds. Seed breeding practice has proved that the hybrid vigor depends on the homozygous degree and combining ability of crossing parents. In production of hybrid corn and hybrid rice, it is often seen that the vigor of hybrid seeds of one strong-vigor hybrid variety is reduced due to low purity of parent seeds.

### SUMMARY

In view of this, the present disclosure provides a method for producing three-line hybrid wheat parent foundation seeds and mainly aims at ensuring the purity and hybrid vigor performance of parent seeds.

For achieving the above purpose, the present disclosure mainly provides the following technical scheme:

in one aspect, embodiments of the present disclosure provide a method for producing three-line hybrid wheat parent foundation seeds, including the following steps:

a. determining whether sterile-line head progeny rows satisfy the 100% sterile degree index of a sterile line or not by bagged selfing of the sterile-line head progeny rows, separately harvesting the sterile-line head progeny rows satisfying the sterile degree index and bagged outcrossed heads of maintainer-line head progeny rows, and mixedly harvesting the sterile-line head progeny rows satisfying the sterile degree index;

b. achieving cyclic breeding of sterile-line spike lines by using the bagged outcrossed head seeds separately harvested in the step a to perform interplanting with the same type of maintainer lines; planting the mixedly harvested sterile-line head progeny row seeds for sterile-line foundation seed plots, and harvesting sterile-line foundation seeds after maturing;

c. conducting pairing, bagging and test crossing on the bagged outcrossed head seeds separately harvested in the step a and restorer-line head progeny rows, correspondingly numbering the restorer-line head progeny rows and bagged test-crossed heads, and separately harvesting bagged test-crossed seeds;

d. conducting yield comparison on the bagged test-crossed seeds separately harvested in the step c, weeding out the test-crossed seeds expressing poor hybrid vigor, and preserving restorer-line head progeny row seeds corresponding to the test-crossed seeds in number; and

e. taking a small amount of preserved restorer-line head progeny row seeds corresponding to the test-crossed seeds meeting the requirement for cyclic breeding of restorer-line spike lines, then mixing the head progeny row seeds for planting restorer-line foundation seed plots, and harvesting them as restorer-line foundation seeds.

Preferably, in the step a, interplanting is conducted on sterile-line head progeny row female parents and the same type of maintainer-line head progeny row male parents according to the row ratio of 2 to 1 in an isolation zone;

after heading, at least one stem head is randomly selected from each sterile-line head progeny row for bagged selfing, and at least two heads are randomly selected from each sterile-line head progeny row for bagged outcrossing with the adjacent maintainer-line heads;

the flowering situations of the sterile-line head progeny rows are observed at the flowering stage;

the fructification situations of the bagged selfed heads of the sterile-line head progeny rows are checked at a maturation stage;

the sterile-line head progeny rows with pollen-spreading plants or bearing bagged selfed fruits do not satisfy the 100% sterile degree index of the sterile line;

the sterile-line head progeny rows which do not satisfy the sterile degree index are weeded out; and

after bagged outcrossed heads in the sterile-line head progeny rows satisfying the sterile degree index are separately harvested, the sterile-line head progeny row seeds satisfying the sterile degree index are mixedly harvested.

The bagged outcrossed heads in the sterile-line head progeny rows are respectively and separately threshed and preserved, at least one of the bagged outcrossed heads in the sterile-line head progeny rows is used for cyclic breeding of the sterile-line spike lines, and at least one head is used for test crossing with restorer-line head progeny rows.

Preferably, the mixedly harvested sterile-line head progeny row seeds and the same type of the maintainer-line male parents are interplanted in a row ratio of 6-10 to 2 in the isolation zone; during the flowering and pollen spreading stage of the maintainer-line, artificial supplementary pollination is continuously performed by using blowers for 5 days, then 2 rows of the maintainer-line male parent plants are cut off; and the sterile-line female parent seeds are harvested at the maturation stage, which are the sterile-line foundation seeds.

Preferably, the step c is specifically as follows:

the sterile-line head progeny row female parents and the restorer-line head progeny row male parents are interplanted by a row ratio of 1 to 2 in the isolation zone, wherein the sterile-line head progeny row female parents are the bagged outcrossed head seeds of the harvested sterile-line head progeny rows satisfying the sterile degree index;

the restorer-line head progeny rows with poor expression are weeded out in fields;

after heading, at least two heads are randomly selected from each sterile-line head progeny row for bagged test crossing with heads of adjacent restorer-line head progeny rows, and the test-crossed heads and corresponding restorer-line head progeny rows are numbered, listed and marked in an one-to-one correspondence mode; and

at the maturation stage, separately harvested bagged test-crossed heads produced by the sterile lines x the restorer lines are respectively preserved according to numbers to obtain test-crossed seeds, then correspondingly numbered restorer-line head progeny rows are harvested, the head progeny rows are separately threshed and are respectively bagged and preserved according to the numbers to obtain restorer-line head progeny row seeds.

Preferably, yield comparison is conducted on the restorer-line test-crossed seeds, and an augmentation test design is adopted for field arrangement with mainly planted commercial variety as a control; the restorer-line test-crossed seeds and the restorer-line head progeny row seeds corresponding to the restorer-line test-crossed seeds remarkably lower than the control in yield are weeded out according to one-to-one corresponding numbers; a small amount of seeds of each selected restorer-line head progeny row is firstly reserved for restoring cyclic breeding of the restorer-line spike lines, and then the selected restorer-line head progeny row seeds are mixed.

Compared with the prior art, the present disclosure has the advantages that:

the method for producing three-line hybrid wheat parent foundation seeds of the embodiments of the present disclosure ensures the 100% sterile degree index and high combining ability of the sterile lines, ensures the purity of parent seeds, ensures the seed quality of strong-vigor hybrid wheat variety parents and accordingly ensures the utilization of the strong-vigor hybrid seeds.

### BRIEF DESCRIPTION OF THE DRAWINGS

FIG. 1 is a flow diagram of an embodiment of the present disclosure.

### DETAILED DESCRIPTION

Detailed description of the present disclosure is further given in combination with the specific embodiment, but is not used for limiting the present disclosure. In the following description, ‘one embodiment’ or different ‘embodiments’ do not always refer to the same embodiment. In addition, the specific features, structures or characteristics of one or more embodiments can be combined in any suitable form.

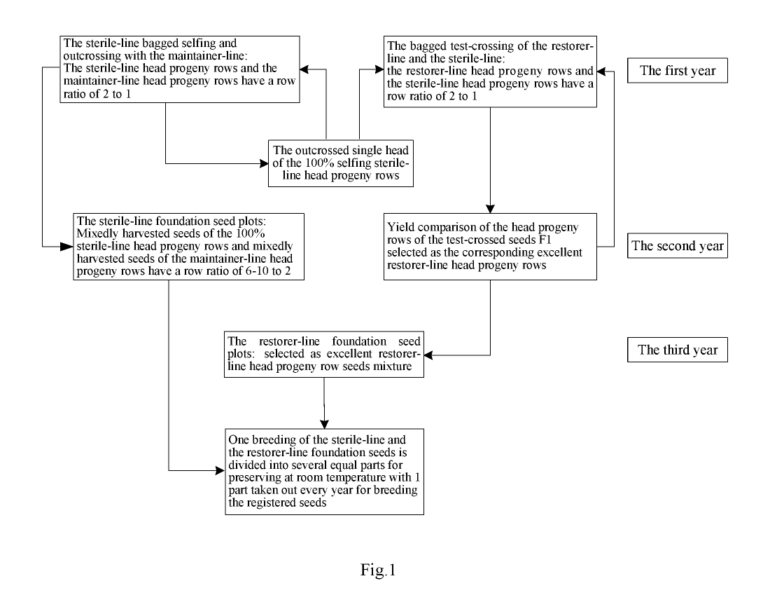

Referring to FIG. 1, a three-line hybrid wheat parent foundation seed production method includes the following steps:

a. determining whether sterile-line head progeny rows satisfy the 100% sterile degree index of a sterile line or not by bagged selfing of the sterile-line head progeny rows, separately harvesting the sterile-line head progeny rows satisfying the sterile degree index and bagged outcrossed heads of maintainer-line head progeny rows, and mixedly harvesting the sterile-line head progeny rows satisfying the sterile degree index;

b. achieving cyclic breeding of sterile-line spike lines by using the bagged outcrossed head seeds separately harvested in the step a to perform interplanting with the same type of maintainer lines; planting the mixedly harvested sterile-line head progeny row seeds for sterile-line foundation seed plots, and harvesting sterile-line foundation seeds after maturing;

c. conducting pairing, bagging and test crossing on the bagged outcrossed head seeds separately harvested in the step a and restorer-line head progeny rows, correspondingly numbering the restorer-line head progeny rows and bagged test-crossed heads, and separately harvesting bagged test-crossed seeds;

d. conducting yield comparison on the bagged test-crossed seeds separately harvested in the step c, weeding out the test-crossed seeds expressing poor hybrid vigor, and preserving restorer-line head progeny row seeds corresponding to the test-crossed seeds in number;

e. taking a small amount of preserved restorer-line head progeny row seeds corresponding to the test-crossed seeds meeting the requirement for cyclic breeding of restorer-line spike lines, then mixing the head progeny row seeds for planting restorer-line foundation seed plots, and harvesting them as restorer-line foundation seeds.

In the method of the embodiment of the present disclosure, interplanting is conducted on the sterile-line head progeny rows and the restorer-line head progeny rows, the sterile degree is identified by bagged selfing of the sterile-line head progeny rows, the sterile-line head progeny rows are bred by using the bagged outcrossed heads of the sterile-line head progeny rows satisfying the sterile degree index, and accordingly the 100% sterile degree index of female-parent sterile lines is ensured. The restorer-line head progeny rows expressing poor hybrid vigor are weeded out by yield comparison conducted on the restorer-line head progeny rows and the bagged test-crossed heads of the bagged test-crossed head progeny rows of the sterile-line head progeny rows satisfying the sterile degree index, the high combining ability of male parent restorer lines is ensured, the purity of parent seeds is ensured, the seed quality of the strong-vigor hybrid wheat variety parents is ensured, and accordingly the utilization of the strong-vigor hybrid seeds is ensured.

Preferably for the above embodiment, in the step a,

the interplanting is conducted on the sterile-line head progeny row female parents and the same type of maintainer-line head progeny row male parents according to the row ratio of 2 to 1 in the isolation zone; certainly, the interplanting can be also performed according to the other row ratios;

after heading, at least one stem head (generally 1 to 2 heads) is randomly selected from each sterile-line head progeny row for bagged selfing, and at least two heads (generally 2 heads) are randomly selected from each sterile-line head progeny row for bagged outcrossing with the adjacent maintainer-line heads;

the flowering situations of the sterile-line head progeny rows are observed at a flowering stage;

the fructification situations of the bagged selfed heads of the sterile-line head progeny rows are checked at a maturation stage;

the sterile-line head progeny rows with pollen-spreading plants or bearing bagged selfed fruits do not satisfy the 100% sterile degree index of the female-parent sterile lines;

the sterile-line head progeny rows which do not satisfy the sterile degree index are weeded out;

after bagged outcrossed heads in the sterile-line head progeny rows satisfying the sterile degree index are separately harvested, the sterile-line head progeny row seeds satisfying the sterile degree index are mixedly harvested.

In the separately harvested bagged outcrossed heads in the sterile-line head progeny rows, one head can be used for cyclic breeding of the sterile-line spike lines, and another one of the others can be used for test crossing with the restorer-line head progeny rows.

In the embodiment of the present disclosure, whether the sterile-line head progeny rows satisfy the sterile degree index of the female-parent sterile lines or not is judged by observing whether the sterile-line head progeny rows contain pollen-spreading plants or the bagged selfed heads bear fruits or not, and the 100% sterile degree index of the sterile-line head progeny rows is fully ensured.

Preferably, the mixedly harvested sterile-line head progeny row seeds and the same type of the maintainer-line male parents are interplanted in a row ratio of 6-10 to 2 in the isolation zone; during the flowering and pollen spreading stage of the maintainer-line, artificial supplementary pollination is continuously performed by using blowers for 5 days, then 2 rows of the maintainer-line male parent plants are cut off; the sterile-line female parent seeds are harvested at the maturation stage, which are the sterile-line foundation seeds. In the embodiment, the fertilization rate is ensured by using the blowers to perform artificial supplementary pollination. The cutoff of the maintainer-line male parent plants facilitates growth and harvesting of sterile-line female parents.

Preferably for the above embodiment, the step c is described in detail as follows:

the sterile-line head progeny row female parents and the restorer-line head progeny row male parents are interplanted by a row ratio of 1 to 2 (without excluding other row ratios) in the isolation zone, wherein the sterile-line head progeny row female parents are selected from the bagged outcrossed head seeds of the harvested sterile-line head progeny rows satisfying the sterile degree index;

the restorer-line head progeny rows with poor expression are weeded out in fields;

after heading, at least two heads (generally two heads) are randomly selected from each sterile-line head progeny row for bagged test crossing with heads of the adjacent restorer-line head progeny rows, and the test-crossed heads and corresponding restorer-line head progeny rows are numbered, listed and marked in an one-to-one correspondence mode;

at the maturation stage, the bagged test-crossed heads of the sterile-line x the restorer-line are separately harvested and are respectively preserved according to numbers to obtain the test-crossed seeds, then the corresponding numbered restorer-line head progeny rows are harvested, the head progeny rows are separately threshed and are respectively packaged and preserved according to numbers to obtain the restorer-line head progeny row seeds. In the embodiment of the present disclosure, the bagged test-crossed heads and the corresponding restorer-line head progeny rows are correspondingly numbered and are respectively harvested and preserved according to the numbers for facilitating follow-up screening and removal of the restorer-line head progeny rows with poor hybrid vigor and ensuring the high combining ability of selected restorer-line head progeny rows.

Preferably for the above embodiment, yield comparison is conducted on the restorer-line test-crossed seeds, and an augmentation test design is adopted for field arrangement with a mainly planted commercial variety as a control; the restorer-line head progeny row seeds corresponding to the restorer-line test-crossed seeds remarkably lower than the control in yield are weeded out according to the one-to-one corresponding numbers; a small amount of seeds of each selected restorer-line head progeny row is firstly reserved for restoring cyclic breeding of the restorer-line spike lines, and then the selected restorer-line head progeny row seeds are mixed.

### Application Examples

A three-line hybrid wheat variety Xindong No. 43 (AL18Ax99AR144-1) bred in the Wheat Department of Crop Research Institute of Xinjiang Academy of Land Reclamation Sciences (Certificate of Variety Examination and Approval: newly approved wheat 201305) is taken as an example. Three-line parents of the variety are sterile lines AL18A, same type of maintainer lines 18B, restorer lines 99AR144-1 respectively called sterile lines, maintainer lines and restorer lines for short. See FIG. 1, three-line parent foundation seed production is performed by adopting the method of the embodiment of the present disclosure:

### 1. Planting of Sterile-Line and Maintainer-Line Head Progeny Rows

Interplanting is conducted on the sterile-line (female-parent) and maintainer-line (male-parent) head progeny rows according to the row ratio of 2 to 1 in the safe isolation zone in which no other wheat varieties are planted within the surrounding 100-meter range. That is, one row of maintainer lines is planted between every two rows of sterile lines. The row length is 2 m, the row spacing is 25 cm, and one head of threshed seed is hill-seeded in each row. Generally, 1000 sterile-line head progeny rows and 500 maintainer-line head progeny rows are needed in the area of 666.7 m². After heading, 1 to 2 stem heads are randomly selected from each sterile-line head progeny row and are covered with a sheepskin bag for selfing pollination after wheat awns are sheared off, and then 2 heads are selected from each sterile-line head progeny row are bagged together with 2 heads of the adjacent maintainer-line head progeny rows in one paper bag for outcross pollinating. The flowering situations of the sterile-line head progeny rows are observed at a flowering stage. The fructification situations of the bagged selfed heads of the sterile-line head progeny rows are checked at a maturation stage. The sterile-line head progeny rows which contain pollen-spreading plants or bear bagged selfed fruits and do not satisfy the 100% sterile degree index of the sterile lines are weeded out. After the bagged outcrossed heads in the selected sterile-line head progeny rows satisfying the 100% sterile degree index are separately harvested, then all the selected sterile-line head progeny row seeds are mixedly harvested, and the 100% sterile degree of the sterile lines is ensured. The maintainer lines are cut off and moved out of the fields after pollination is completed.

### 2. Utilization of Sterile-Line Outcrossed Heads

Two bagged outcrossed heads in the sterile-line head progeny rows are respectively and separately threshed and preserved, one head is used for cyclic breeding of the sterile-line spike lines, and another one is used for test crossing with the restorer-line heads.

### 3. Production of Sterile Line Foundation Seeds

The mixedly harvested sterile-line head progeny row seeds and the maintainer lines are mechanically sowed in the isolation zone according to the row ratio of 10 to 2. That is, 2 rows of maintainer lines are planted between every adjacent 10 rows of sterile lines. The average row spacing is 15 cm. When the row spacing of a sower is adjusted, the row spacing (20 cm) between male parent rows and adjacent female parent rows is slightly increased for distinguishing joint row boundaries of the male parents and the female parents, and the slightly wide row spacing facilitates the male parent rows cutoff after pollination. The sowing quantity is calculated based on 300 thousand basic seedlings. Weeding and rouging are strictly performed after heading. During flowering and pollen spreading of the maintainer lines, artificial supplementary pollination is continuously performed by using blowers for 5 days, then 2 rows of maintainer-line male parent plants are cut off. The sterile-line seeds are harvested at the maturation stage, separated harvesting, separated transportation, separated sun-drying, separated threshing and special storage are strictly carried out, and sterile-line foundation seeds are obtained.

### 4. Planting the Restorer-Line Head Progeny Rows and the Sterile-Line Test-Crossed Head Progeny Rows

In the isolation zone, the head progeny rows from the sterile-line outcrossed head progeny rows (female parent) and the restorer-line head progeny rows (male parent) are planted in a row ratio of 1 to 2. That is, one row of the sterile-line head progeny rows is planted between every two rows of the restorer-line head progeny rows. The row length is 2 m, the row spacing is 25 cm, and one head of threshed seeds is hill-seeded in each row. Generally, 1000 sterile-line head progeny rows and 500 maintainer-line head progeny rows are needed in the area of 666.7 m². By observing the typicality and head progeny rows consistency of the restorer-line in fields, the restorer-line head progeny rows with poor expressions and inconsistency are weeded out. After heading, 2 heads selected from each sterile-line head progeny row are bagged together with 2 heads of the adjacent restorer-line head progeny rows in one paper bag for outcross pollinating, that is the test crossing, and the cost may be reduced by bagging 4 heads in one bag. Also, 1 head of the sterile-line head progeny rows can be bagged together with 1 head of the adjacent restorer-line head progeny rows in one paper bag for outcross pollinating. The test-crossed heads and the restorer-line head progeny rows are numbered, listed and marked in a one-to-one correspondence. At the maturation stage, the bagged test-crossed heads are separately harvested at first, then the correspondingly numbered restorer-line head progeny rows are harvested, and the heads rows are respectively threshed and packaged in seeds bags for safekeeping. The sterile-line head progeny rows are mixedly harvested, i.e. the hybrid seeds.

### 5. Comparing the Sterile-Line x the Restorer-Line Test-Crossed Seeds

Yield comparison is conducted on the harvested test-crossed seeds. The augmentation test design is adopted for field arrangement with a mainly planted commercial variety as a control. Each test-crossed seed is planted in 2 rows with rows length of 2 m and rows width of 25 cm, and each row is sparsely sowed with the threshed seeds of the test-crossed heads. The test-crossed seeds are harvested at the maturation stage and are threshed and weighed according to their numbers; and the test-crossed seeds, which are significantly lower than the preserved seeds of the correspondingly numbered restorer-line head progeny rows of the control in yield according to the statistical analysis, are weeded out. A small amount of seeds of each selected restorer-line head progeny row (approximately 80 grains) are firstly reserved for cyclic breeding of the restorer-line spike lines, and then the selected restorer-line head progeny row seeds are mixed.

### 6. Foundation Seeds Production of the Restorer-Line Species

Fields with good soil conditions are selected, the mixed seeds of the last year restorer-line head progeny rows are mechanically sowed in drills with an average row spacing of 15 cm, and the sowing quantity are calculated by 300,000 basic seedlings. The fields are finely managed, and by rigorously rogued in two times after heading and with strictly separated harvesting, separated transportation, separated sun-drying, separated threshing and special preservation at the maturation stage, the restorer-line foundation seeds can be obtained.

### 7. Preservation and Utilization of the Foundation Seeds

The produced sterile-line foundation seeds and restorer-line foundation seeds are separately divided into 5 equal parts and are precisely marked, followed by preserving in a dry and ventilated seed bank at room temperature, and every year 1 part is separately taken out for producing the sterile-line registered seeds and the restorer-line registered seeds.

The above description are merely embodiments of the present disclosure which is not intended to limit the protection scope of the present disclosure, and it will be apparent to those skilled in the art that modifications and changes may be readily made within the disclosure of the disclosure and without departing from the scope of the disclosure. Thus, the protection scope of the present disclosure shall be subject to the protection scope defined by the claims.

What is claimed is:

1. A method for producing three-line hybrid wheat parent foundation seeds, comprising the following steps:
a. determining whether sterile-line head progeny rows satisfy the 100% sterile degree index of a sterile line or not by bagged selfing of the sterile-line head progeny rows, separately harvesting the sterile-line head progeny rows satisfying the sterile degree index and bagged outcrossed heads of maintainer-line head progeny rows, and mixedly harvesting the sterile-line head progeny rows satisfying the sterile degree index;
b. achieving cyclic breeding of sterile-line spike lines by using the bagged outcrossed head seeds separately harvested in the step a to perform interplanting with the same type of maintainer lines; planting the mixedly harvested sterile-line head progeny row seeds for sterile-line foundation seed plots, and harvesting sterile-line foundation seeds after maturing;
c. conducting pairing, bagging and test crossing on the bagged outcrossed head seeds separately harvested in the step a and restorer-line head progeny rows, correspondingly numbering the restorer-line head progeny rows and bagged test-crossed heads, and separately harvesting bagged test-crossed seeds;
d. conducting yield comparison on the bagged test-crossed seeds separately harvested in the step c, weeding out the test-crossed seeds expressing poor hybrid vigor, and preserving restorer-line head progeny row seeds corresponding to the test-crossed seeds in number; and
e. taking a small amount of preserved restorer-line head progeny row seeds corresponding to the test-crossed seeds meeting the requirement for cyclic breeding of restorer-line spike lines, then mixing the head progeny row seeds for planting restorer-line foundation seed plots, and harvesting them as restorer-line foundation seeds.

2. The method according to claim 1, wherein in the step a,
interplanting is conducted on sterile-line head progeny row female parents and the same type of maintainer-line head progeny row male parents according to the row ratio of 2 to 1 in an isolation zone;
after heading, at least one stem head is randomly selected from each sterile-line head progeny row for bagged selfing, and at least two heads are randomly selected from each sterile-line head progeny row for bagged outcrossing with the adjacent maintainer-line heads;
the flowering situations of the sterile-line head progeny rows are observed at the flowering stage;
the fructification situations of the bagged selfed heads of the sterile-line head progeny rows are checked at the maturation stage;
the sterile-line head progeny rows with pollen-spreading plants or bearing bagged selfed fruits do not satisfy the 100% sterile degree index of the female-parent sterile lines;
the sterile-line head progeny rows which do not satisfy the sterile degree index are weeded out; and
after the bagged outcrossed heads in the sterile-line head progeny rows satisfying the sterile degree index are separately harvested, the sterile-line head progeny row seeds satisfying the sterile degree index are mixedly harvested.

3. The method according to claim 2, wherein the method further comprises: the mixedly harvested sterile-line head progeny row seeds and the same type of the maintainer-line male parents are interplanted in a row ratio of 6-10 to 2 in the isolation zone; during the flowering and pollen spreading stage of the maintainer-line, artificial supplementary pollination is continuously performed by using blowers for 5 days, then 2 rows of the maintainer-line male parent plants are cut off; and the sterile-line female parent seeds are harvested at the maturation stage, which are the sterile-line foundation seeds.

4. The method according to claim 1, wherein the step c comprises:
the sterile-line head progeny row female parents and the restorer-line head progeny row male parents are interplanted by a row ratio of 1 to 2 in the isolation zone, wherein the sterile-line head progeny row female parents are the bagged outcrossed head seeds of the harvested sterile-line head progeny rows satisfying the sterile degree index;
the restorer-line head progeny rows with poor expression are weeded out in fields;
after heading, at least two heads are randomly selected from each sterile-line head progeny row for bagged outcrossing with heads of the adjacent restorer-line head progeny rows, and the test-crossed heads and the corresponding restorer-line head progeny rows are numbered, listed and marked in an one-to-one correspondence; and
at the maturation stage, the bagged test-crossed heads of the sterile-line x the restorer-line are separately harvested and are respectively preserved according to numbers to obtain the test-crossed seeds, then the corresponding numbered restorer-line head progeny rows are harvested, and the head progeny rows are separately threshed and are respectively packaged and preserved according to numbers to obtain the restorer-line head progeny row seeds.

5. The method according to claim 1, wherein the method further comprises: yield comparison is conducted on the restorer-line test-crossed seeds and an augmentation test design is adopted for field arrangement with a mainly planted commercial variety as a control; the restorer-line head progeny row seeds corresponding to the restorer-line test-crossed seeds remarkably lower than the control in yield are weeded out according to the one-to-one corresponding numbers to preserve seeds; and a small amount of seeds of each selected restorer-line head progeny row is firstly reserved for restoring cyclic breeding of the restorer-line spike lines, and then the selected restorer-line head progeny row seeds are mixed for planting the restorer-line foundation seeds.
